(19) United States
(12) Patent Application Publication
(10) Pub. No.: US 2006/0150260 A1
(43) Pub. Date: Jul. 6, 2006

(54) Genes related to drug resistance

(75) Inventors: Vivtor V. Levenson, Chicago, IL (US); Natalie A. Motchoulskala, Schaumburg, IL (US)

Correspondence Address: MEDLEN & CARROLL, LLP;Suite 350, 101 Howard Street, San Francisco, CA, 94105, US

(73) Assignee: Northwestern University, Evanston, IL (US)

(21) Appl. No.: 11/261,127

(22) Filed: Oct. 28, 2005

Related U.S. Application Data
(60) Provisional application No. 60/622,857, filed on Oct. 28, 2004.

(51) Int. Cl. A01K 67/027; C12Q 1/68

(52) U.S. Cl. 800/3; 800/14; 435/6

(57) ABSTRACT

The present invention relates to genetic profiles and markers of cancers and provides systems and methods for screening drugs that are effective for specific patients and types of cancers.

This application claims priority to provisional patent application Ser. No. 60/622,857, filed Oct. 28, 2004, which is herein incorporated by reference in its entirety.

### FIELD OF THE INVENTION

The present invention relates to genetic profiles and markers of cancers and provides systems and methods for screening drugs that are effective for specific patients and types of cancers.

### BACKGROUND OF THE INVENTION

The efficacy of anti-cancer drugs varies widely among individual patients. A large proportion of cancer patients suffer adverse effects from chemotherapy while showing no effective response in terms of tumor regression. Furthermore, many patients initially respond well to treatment but eventually develop resistance to the treatment. For example, Tamoxifen is the most extensively used hormonal treatment for all stages of breast cancer and has recently been approved for the prevention of breast cancer in high-risk women (O'Regan et al., The Lancet Oncology, 2002, 3, 207-214). In the vast majority of cases, however, even initially sensitive patients develop resistance to the drug, making identification of putative resistance genes an important medical challenge (McGregor-Schafer et al., J. Steroid Biochem & Mol Biol, 2002, 83, 75-83; de Cremoux et al., Endoc-Rel. Cancer, 2003, 10, 409-418; Brockdorffet al., Endoc-Rel Cancer, 2003, 10, 579-590; Clarke et al., Oncogene, 2003, 22, 7316-7339). Properties of cancer cells are determined by complicated interactions among all gene products expressed in cancer cells, and it is certain that many proteins, including enzymes involved in apoptosis, DNA repair, and metabolism and detoxification of drugs, affect individual responses. Hence, to distinguish responders from non-responders before starting treatment, i.e., to offer a “personalized” program of more effective chemotherapy, to relieve patients from unnecessary side effects, and to identify putative drug resistance genes, a larger set of genes should be identified to serve as accurate predictive markers. Additionally, identification of genes responsible for drug resistance is needed to provide biomarkers that can be used to monitor the development of resistance, and to provide drug targets to block or reverse the resistance process or to provide substitute, effective therapies.

### SUMMARY OF THE INVENTION

The present invention relates to genetic profiles and markers of cancers and provides systems and methods for screening drugs that are effective for specific patients and types of cancers. Accordingly, in some embodiments, the present invention provides resistance inducing genes and methods for reducing resistance of cells and subjects to chemotherapy comprising inhibiting the expression or biological activity of the resistance inducing genes. The present invention further provides diagnostic and research methods to identify individuals resistant to chemotherapy and test compounds for their ability to inhibit the function of resistance inducing genes.

For example, in some embodiments, the present invention provides a method of sensitizing cells to chemotherapeutic agents, the method comprising inhibiting the expression of a resistance inducing gene (e.g., macrophage migration inhibitory factor (MIF), prolylcarboxypeptidase, tRNA-guanine transglycosylase, or kinesin light chain (KNS2)). In some embodiments, the inhibiting the expression of a resistance inducing gene comprises introducing an antisense or siRNA complementary to the resistance inducing gene into the cell. In other embodiments, inhibiting the expression of a resistance inducing gene comprises introducing an antibody that specifically binds to a protein encoded by the resistance inducing gene into the cell. In still further embodiments, inhibiting the expression of a resistance inducing gene comprises introducing a small molecule therapeutic that inhibits the expression or biological activity of the resistance inducing gene into the cell. In some embodiments, the chemotherapeutic agent is tamoxifen or 4-hydroxytamoxifen. In some embodiments, the cell is in vitro. In other embodiments, the cell is in vivo (e.g., in an organism including, but not limited to, a non-human mammal and a human).

The present invention further provides a a method of monitoring chemotherapeutic treatment, the method comprising measuring the expression of a resistance inducing gene (e.g., macrophage migration inhibitory factor (MIF), prolylcarboxypeptidase, tRNA-guanine transglycosylase, or kinesin light chain (KNS2)) in a sample obtained from a subject undergoing chemotherapy. In some embodiments, measuring the expression of a resistance inducing gene comprises exposing the sample to a nucleic acid complementary to the resistance inducing gene. In other embodiments, measuring the expression of a resistance inducing gene comprises exposing the sample to an antibody that specifically binds to a polypeptide encoded by the resistance inducing gene. In some embodiments, the chemotherapeutic treatment is tamoxifen or 4-hydroxytamoxifen.

In still further embodiments, the present invention provides a method of screening compounds, comprising: providing a cell expressing a resistance inducing gene (e.g., macrophage migration inhibitory factor (MIF), prolylcarboxypeptidase, tRNA-guanine transglycosylase, or kinesin light chain (KNS2)); and exposing the cell to a test compound. In some embodiments, the method further comprises the step of measuring the effect of the test compound on the level of expression of the resistance inducing gene. In some embodiments, the test compound is an antisense nucleic acid complementary to the resistance inducing gene, an siRNA complementary to the resistance inducing gene, an antibody that specifically hybridizes to a polypeptide encoded by the resistance inducing gene, or a small molecule therapeutic. In some embodiments, the cell is in vitro. In other embodiments, the cell is in vivo (e.g., in a non-human mammal).

In yet other embodiments, the present invention provides a method of detecting efficacy of chemotherapeutic agents comprising detecting the expression or activity of a marker (e.g., macrophage migration inhibitory factor (MIF), prolylcarboxypeptidase, tRNA-guanine transglycosylase, or kinesin light chain (KNS2)). In some embodiments, the chemotherapeutic treatment is tamoxifen or 4-hydroxytamoxifen.

### DESCRIPTION OF THE FIGURES

FIG. 1 shows the identification of cDNA inserts in surviving MCF-7 clones. FIG. 1A shows positioning of the vector-specific primers used for cDNA insert recovery. FIG. 1B shows cDNA inserts recovered from clones B4 (lane 2), B6 (lane 3), D10 (lane 6) and ES (lane 7). Amplification of GFP (lane 5) served as a control. FIG. 1C shows alignment of 5′ ends of recovered clones with corresponding GenBank entries (numbers in parenthesis).

FIG. 2 shows the re-introduction of identified cDNA inserts made MCF-7 cells resistant to 4OHTAM. FIG. 2A shows integration of corresponding cDNA inserts confirmed by PCR using genomic DNA from re-infected populations. FIG. 2B shows that a colony formation assay following 4OHTAM treatment indicated increased survival of populations, infected with corresponding constructs expressing cDNA inserts. FIG. 2C shows that a quantitative assessment of colony formation assay showed substantial survival advantage for cDNA-containing populations (˜65% survival of populations compared to >10% survival of GFP-expressing control at 7.5 mM of 40HTAM).

FIG. 3 shows cell growth characteristics in RIGs-expressing populations. FIG. 3A shows that without 4OHTAM RIGs-containing cells grew faster than parental MCF7 or control GFP-expressing cells. FIG. 3B shows that growth of RIGs-containing and parental cells was inhibited by 7.5 mM 4OHTAM. FIG. 3C shows that RIGs-containing cells either continued to grow (B4, B6 and D10) or survived treatment with 10 mM 4OHTAM (ES), while proliferation of MCF-7 cells was blocked.

FIG. 4 shows that RIGs enhance cell viability in drug-free conditions and when treated with 4OHTAM. FIG. 4A shows that in drug-free media RIGs do no affect cell cycle distribution, although they reduce cell debris (cells with DNA content of less than 1 n). FIG. 4B shows that cells with RIGs respond to 4OHTAM (10 mM) by partial G1 phase block (increased fraction of cells in G1 phase and decreased fraction of cells in S phase compared to parental MCF-7 cells).

FIG. 5 shows that cell death caused by 4OHTAM in MCF-7 cells does not have characteristic features of apoptosis. FIG. 5A shows that no apoptotic subG1 peak was observed in 4OHTAM-treated cells. FIG. 5B shows that all four RIGs-containing cell populations showed significantly lower accumulation of cell debris compared to parental MCF7 cells. FIG. 5C shows agarose gel electrophoresis of DNA isolated from untreated cells (lane 1) and cells after treatment with 10 mM (lane 2) and 20 mM (lane 3) 4OHTAM did not display characteristic nucleosomal DNA ladder. M—DNA marker, Co—control apoptotic ladder.

FIG. 6 shows that 4OHTAM-induced vacuolization in drug-sensitive and—resistant cells. FIG. 6A shows that drug-sensitive (MCF-7) and drug-resistant cells (B6) in drug-free conditions do not show significant microstructures. FIG. 6B shows that cells treated with 10 μM 4OHTAM (48 hr) display extensive microstructures that correspond to acidic vesicular organelles stained with LysoTracker Blue DND-22 (arrows) in all cases regardless of cell sensitivity to 4OHTAM. X150. FIG. 6C shows cells treated with 10 mM 4OHTAM for different time were stained with LysoTracker Blue DND-22, their fluorescence was measured, and median values were plotted for each cell population.

FIG. 7 shows FACS analysis of cell survival, accumulation of acidic vesicular organelles, mitochondrial survival and functionality in the course of incubation with 10 μM 4OHTAM. FIG. 7A shows double staining with propidium iodide and LysoTracker Blue DND-22 shows that the majority of resistant cells stain highly for acidic vesicular organelles but their plasma membrane remains intact. Upper panel—time course of plasma membrane permeability and accumulation of acidic vesicular organelles during treatment with 10 μM 4OHTAM. Lower panel—distribution of PI-negative cells stained with LysoTracker for different RIGs-expressing populations. FIG. 7B shows that double staining with MitoFluor 589 and LysoTracker Blue DND-22 shows good survival of mitochondria in resistant cells stained highly for acidic vesicular organelles. Upper panel—time course of mitochondrial survival and accumulation of acidic vesicular organelles during treatment with 10 μM 4OHTAM. Lower panel—distribution of cells with high mitochondrial content stained with LysoTracker for different RIGs-expressing populations. FIG. 7C shows that staining with MitoTracker Red CMXRos reveals high functional integrity of mitochondria in resistant cells during treatment with 4OHTAM. Upper panel—time course of mitochondrial activity during treatment with 10 mM 4OHTAM. Lower panel—distribution of cells with low mitochondrial activity for different RIGs-expressing populations.

### DEFINITIONS

To facilitate an understanding of the present invention, a number of terms and phrases are defined below:

As used herein, the term “subject” refers to any animal (e.g., a mammal), including, but not limited to, humans, non-human primates, vertebrates, pigs, rodents, and the like, which is to be the recipient of a particular treatment. Typically, the terms “subject” and “patient” are used interchangeably herein in reference to a human subject.

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “resistance inducing gene” refers to a gene whose expression level, alone or in combination with other genes, is correlated with resistance to a therapeutic agent (e.g., chemotherapy agent). Resistance inducing gene expression may be characterized using any suitable method, including but not limited to, those described in the illustrative Examples below.

As used herein, the term “a reagent that specifically detects expression levels” refers to reagents used to detect the expression of one or more genes (e.g., including but not limited to, the resistance inducing genes of the present invention). Examples of suitable reagents include but are not limited to, nucleic acid probes capable of specifically hybridizing to the gene of interest, PCR primers capable of specifically amplifying the gene of interest, and antibodies capable of specifically binding to proteins expressed by the gene of interest. Other non-limiting examples can be found in the description and examples below.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCi, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk—cell lines, the CAD gene that is used in conjunction with CAD-deficient cells and the mammalian hypoxanthine-guanine phosphoribosyl transferase (hprt) gene that is used in conjunction with hprt—cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp.16.9-16.15.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION OF THE INVENTION

The present invention relates to genetic profiles and markers of cancers and provides systems and methods for screening drugs that are effective for specific patients and types of cancers. Certain preferred embodiments are provided below to illustrate features of the present invention.

Tamoxifen is the most extensively used hormonal treatment for all stages of breast cancer and has recently been approved for the prevention of breast cancer in high-risk women (Regan and Jordan. The Lancet Oncology, 2002, 3, 207-214). In the vast majority of cases, however, even initially sensitive patients develop resistance to the drug, making identification of putative resistance genes an important medical challenge (McGregor-Schafer et al., J. Steroid Biochem & Mol Biol, 2002, 83, 75-83; de Cremoux et al. Endoc-Rel. Cancer, 2003, 10, 409-418; Brockdorffet al., Endoc-Rel Cancer, 2003, 10, 579-590; Clarke et al. Oncogene, 2003, 22, 7316-7339). An alternative model for identification of these genes was developed, applying functional expression selection for survival in the presence of 4-hydroxytamoxifen to estrogen receptor-positive MCF7 cells in the presence of physiological concentrations of estrogen.

To identify genes that can induce resistance to TAM, full-length cDNA expression libraries in retroviral vectors were introduced into naïve MCF-7 cells followed by expression selection screens against TAM in estrogen-containing growth media. Cells that formed clones after exposure to 4OHTAM were used to isolate retroviral inserts, which were re-cloned and individually tested in naïve MCF-7 cells.

Cells carrying several individual inserts—but not parental cells—could grow in the presence of 7.5 μM OHTAM. In drug-free media re-infected cell populations grew much faster than parental cells, while in the presence of drug these populations were significantly more viable. Application of OHTAM caused a substantial S-to-G0/G1 shift in insert-carrying populations compared to parental cells, while accumulation of apoptotic cells (subG0 peak) was notably reduced in TAM-resistant populations. A dramatic increase of the mitochondrial potential was observed in resistant cell populations as compared to MCF-7 after application of 10 μM OHTAM. Changes in GSH content suggest that selected inserts increase efficiency of detoxification by GSH. Interestingly, for all resistant populations OHTAM-induced physiological response was observed much earlier than in MCF-7 cells. Observed changes in cells carrying the selected genes suggest an overall increase in resistance level induced by overexpression of individual genes.

An expression selection screen was performed for genes that protect MCF7 cells from the cytotoxic effects of 4OH-TAM when this drug is applied in estrogen-containing environment. The screen identified several cDNAs that play a role in drug resistance and that provide targets to block or reverse the drug resistance process. For example, the screen identified a cytokine (B4), a member of serine proteinase family (B6), a cellular motor protein (E5), and a tRNA modifying enzyme (D10).

Experiments conducted during the course of development of the present invention used a functional selection screen to isolate genes that convey resistance to cytotoxic action of 4OHTAM. Parental cells used in the study (MCF-7) express estrogen receptor (ER) and respond to estrogen in many different ways (Levenson and Jordan, 1997. Cancer Res 57:3071-3078; Doisneau-Sixou et al., 2003. Endocr Relat Cancer 10:179-186). In clinical practice a certain level of estrogen is present even in postmenopausal women (Purohit and Reed, 2002. Steroids 67:979-983), and TAM treatment of breast cancer patients and concomitant emergence of resistance to the drug take place in the presence of this hormone.

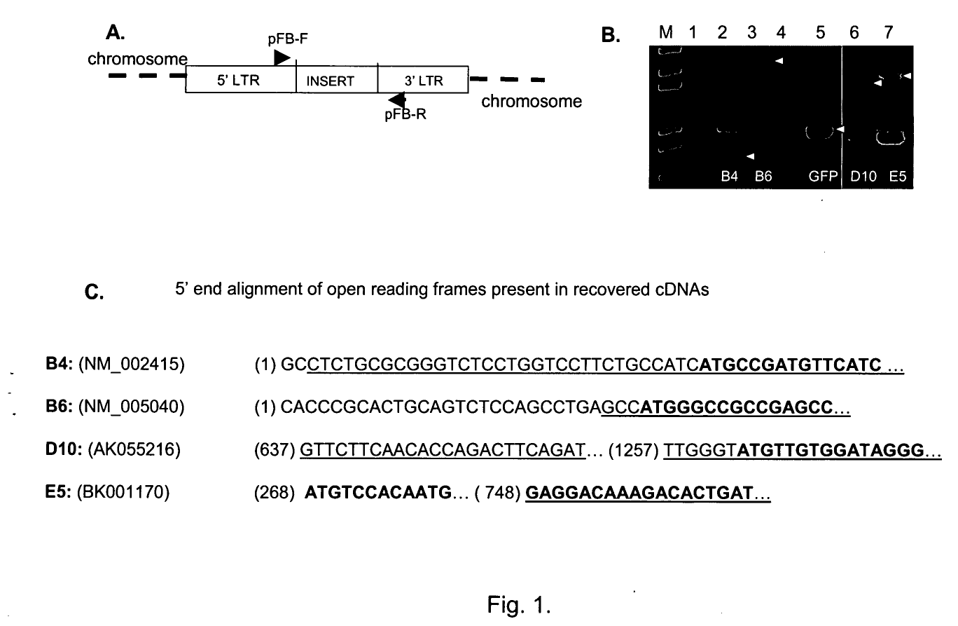

Resistance-inducing genes (RIGs, FIG. 1) recovered after selection contain complete (B4, B6, and D10) or substantial parts (E5) of the corresponding protein-coding regions (FIG. 1C). A PCR assay revealed that a full-length copy of kinesin light chain (KLC1G/KNS2) cDNA was present in the expression library, so 5′ truncation of E5 most likely occurred during retroviral integration (Varmus, 1988. Science 240:1427-1435) and did not reflect shortcomings in library preparation.

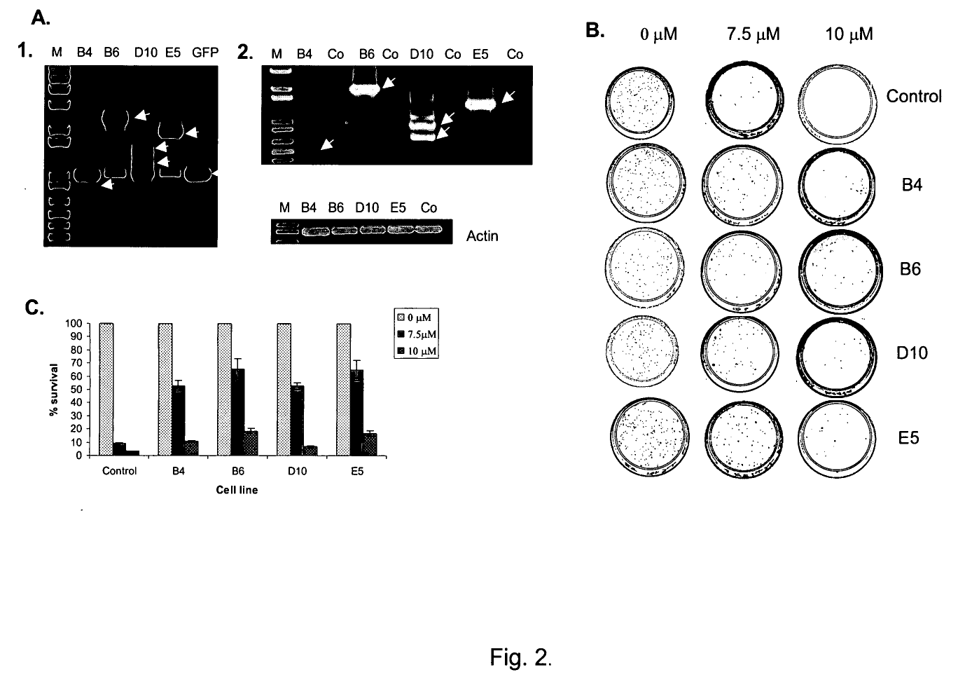

To confirm protective effects of selected RIGs, they were recovered from surviving cellular clones by PCR with vector-specific primers (FIG. 1A), re-cloned into the initial pFB vector, and used to introduce individual RIGs into naïve populations of MCF-7 cells. This step allowed for the avoidance of potential interference from ill-defined genomic mutations induced in surviving cellular clones by drug exposure. To avoid effects of clonal variability, populations of infected cells, rather than single cell clones were used for downstream testing. FIG. 2 shows that the RIGs-infected populations survived 4OHTAM treatment much better than GFP-expressing MCF-7 controls.

Macrophage migration inhibitory factor (MIF) gene encodes a lymphokine involved in cell-mediated immunity, immunoregulation, and inflammation (Nishihira, 2000. J Interferon Cytokine Res 20:751-762). Besides signaling functions of a cytokine, MIF is an oxidoreductase (Kleemann et al., 1998. J Mol Biol 280:85-102) and participates in regulating oxidative cell stress (Nguyen et al., 2003. J Immunol 170:3337-3347). MIF also has D-dopachrome tautomerase activity (Rosengren et al., 1996. Mol Med 2:143-149), which can be blocked by S-hexylglutathione (Swope et al., 1998. J Biol Chem 273:14877-14884), and plays a role in detoxification of toxic quinone products (dopaminechrome and norepinephrinechrome) of the neurotransmitters dopamine and norepinephrine (Matsunaga et al., 1999. J Biol Chem 274:3268-3271).

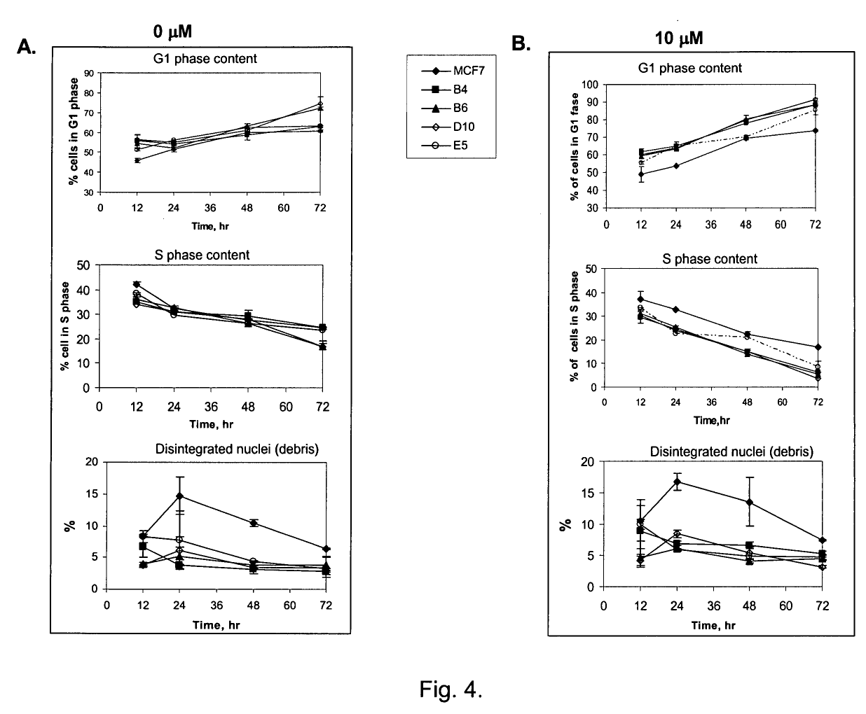

MIF regulates expression of several genes via both MAPK-dependent and—independent pathways (Santos et al., 2004. J Rheumatol 31:1038-1043), and sequestering JAB1, prevents activation of c-jun kinase (JNK) (Kleemann et al., 2000. Nature 408:211-216). While increased expression of MIF correlates with increased growth of murine colon carcinoma (Takahashi et al., 1998. Mol Med 4:707-714), human gastric epithelium (Xia et al., 2005. World J Gastroenterol 11: 1946-1950), and breast cancer (Bando et al., 2002. Jpn J Cancer Res 93:389-396), MIF also induces accumulation of cell cycle inhibitor p27Kip1 (Kleemann et al., 2000. Nature 408:211-216). The present invention is not limited to a particular mechanism. Indeed, an understanding of the mechanism is not necessary to practice the present invention. Nonetheless, it is contemplated that this interplay of pro- and anti-proliferation activity explains negative regulation of MIF expression by proliferation-promoting concentrations of estrogen (Ashcroft et al., 2003. J Clin Invest 111:1309-1318) as well as the results regarding accumulation of MIF-expressing cells in G1 phase after treatment with 4OHTAM (FIG. 4). It is further contemplated that the high level of oxidoreductase activity makes MIF-expressing cells more resistant to 4OHTAM-induced oxidative damage (Gundimeda et al., 1996. J Biol Chem 271:13504-13514) while MIF-dependent stabilization of p27Kip1 delays DNA synthesis, and allows sufficient time for damage repair without induction of cell death. MIF counteracts p53-mediated growth arrest (Hudson et al., 1999. J Exp Med 190:1375-1382; Mitchell et al., 2002. Proc Natl Acad Sci U S A 99:345-350), which is prone to elicit cell death (Urturro et al., 2001. Leukemia 15:1225-1231). Thus, MIF enhances survival-promoting cell cycle block (p27Kip1) and reduces the chances of death-inducing cell cycle arrest (p53).

Prolylcarboxypeptidase (angiotensinase C) (PRCP) is a lysosomal prolylcarboxypeptidase, which cleaves C-terminal amino acids linked to proline in peptides angiotensin II, III and des-Arg9-bradykinin (Odya et al., 1978. J Biol Chem 253:5927-5931), and activates prekallikrein (Shariat-Madar et al., J Biol Chem 277:17962-17969).

The eukaryotic tRNA:guanine transglycosylase (QTRT1/TGT) catalyses the base-for-base exchange of guanine for queuine—a nutrition factor for eukaryotes—at position 34 of the anticodon of tRNAsGUN (where ‘N’ represents one of the four canonical tRNA nucleosides), yielding the modified tRNA nucleoside queuosine (Q) (Langgut and Reisser, 1995. Nucleic Acids Res 23:2488-2491). This unique tRNA modification process was investigated in HeLa cells grown under either aerobic (21% O2) or hypoxic conditions (7% O2) after addition of chemically synthesized queuine to queuine-deficient cells. While the queuine was always inserted into tRNA under aerobic conditions, HeLa cells lost this ability under hypoxic conditions when serum factors became depleted from the culture medium. The activity of the QTRT1/TGT enzyme was restored after treatment of the cells with the protein kinase C activator, TPA, even in the presence of mRNA or protein synthesis inhibitors. The results indicate that the eukaryotic tRNA modifying enzyme, QTRT1/TGT, is a downstream target of activated protein kinase C (Langgut and Reisser, 1995, supra), which is contemplated to explain TAM resistance of MCF-7 cells overexpressing PKC (Tonetti et al., 2000. Br J Cancer 83:782-791; Fournier et al., 2001. Gynecol Oncol 81:366-372; Nabha et al., 2005. Oncogene 24:3166-3176).

Elevated QTRT1/TGT expression has been detected in leukemic cells, and in colon cancer cells and tissues. Induction of differentiation caused a marked decrease in its expression (Ishiwata et al., 2004. Cancer Lett 212:113-119). At the same time the level of the QTRT1/TGT substrate (guanine-containing tRNA) was higher in lung cancer compared to normal lung tissues, suggesting that lower activity of QTRT1/TGT correlates with a neoplastic process (Lo et al., 1992. Anticancer Res 12:1989-1994). Mitochondrial tRNA can be fully modified in normal liver, while in hepatoma 5123D corresponding tRNA is completely unmodified (Randerath et al., 1984. Cancer Res 44:1167-1171). The present invention is not limited to a particular mechanism. Indeed, an understanding of the mechanism is not necessary to practice the present invention. Nonetheless, it is contemplated that with the role of mitochondria in cell death firmly established, it is contemplated that QTRT1/TGT or its products play a role in mitochondrial stabilization and/or regulation of intracellular Ca2+pool.

Kinesin light chain (KLC1G/KNS2) belongs to kinesin motor protein, a tetramer containing two heavy and two light chain proteins. The recovered fragment (E5) lacks 160 aminoacids from the N-terminus of the protein, where the binding site for the heavy chain is located (Diefenbach et al., 1998. Biochemistry 37:16663-16670). Thus, the effects of the E5 RIG are unrelated to its interactions with the heavy chain of kinesin. KLC1G/KNS2 contains the tetratricopeptide repeat, which is involved in various protein-protein interactions (Blatch and Lassle, 1999. Bioessays 21:932-939), and is preserved in E5.

Besides its major function as an intracellular motor kinesin participates in a number of other reactions, including induction of apoptosis via activation of Bax (Tao et al., 2005. Cancer Cell 8:49-59), which may directly relate to its activity as a RIG. Closely related protein Kif1C has been implicated in resistance to anthrax lethal factor (Watters et al., 2001. Curr Biol 11: 1503-1511), while overexpression of kinesin heavy chain has been linked to resistance to etoposide (Axenovich et al., 1998. Cancer Res 58:3423-3428). While mechanisms for these resistance effects are largely unknown, interaction of kinesin with various signaling proteins (Nagata et al., 1998. Embo J 17:149-158; Domer et al., 1999. J Biol Chem 274:33654-33660; Ichimura et al., 2002. Biochemistry 41:5566-5572; Inomata et al., 2003. J Biol Chem 278:22946-22955; Nguyen et al., 2005 J Biol Chem 280:30185-30191), possibly through the tetratricopeptide repeat, indicates that its role might be substantially more complex than just a motor protein.

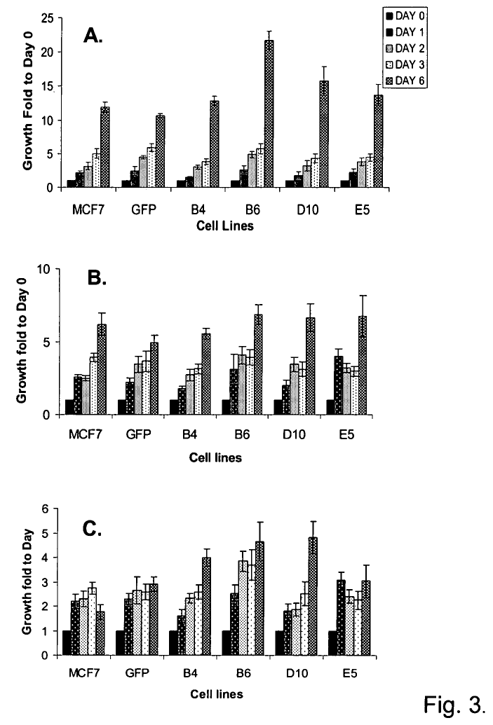

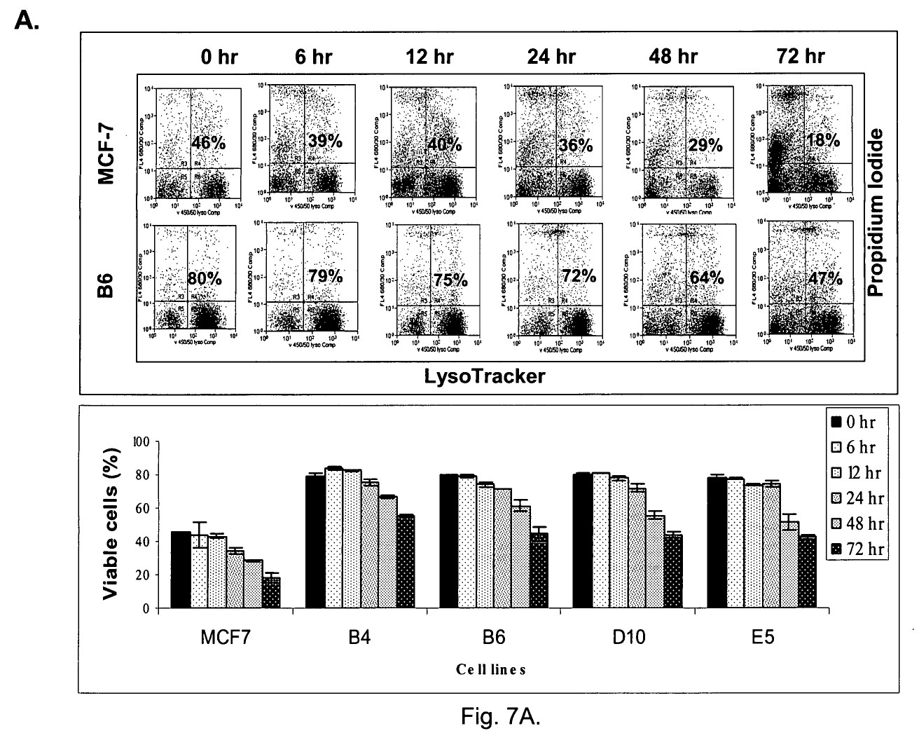

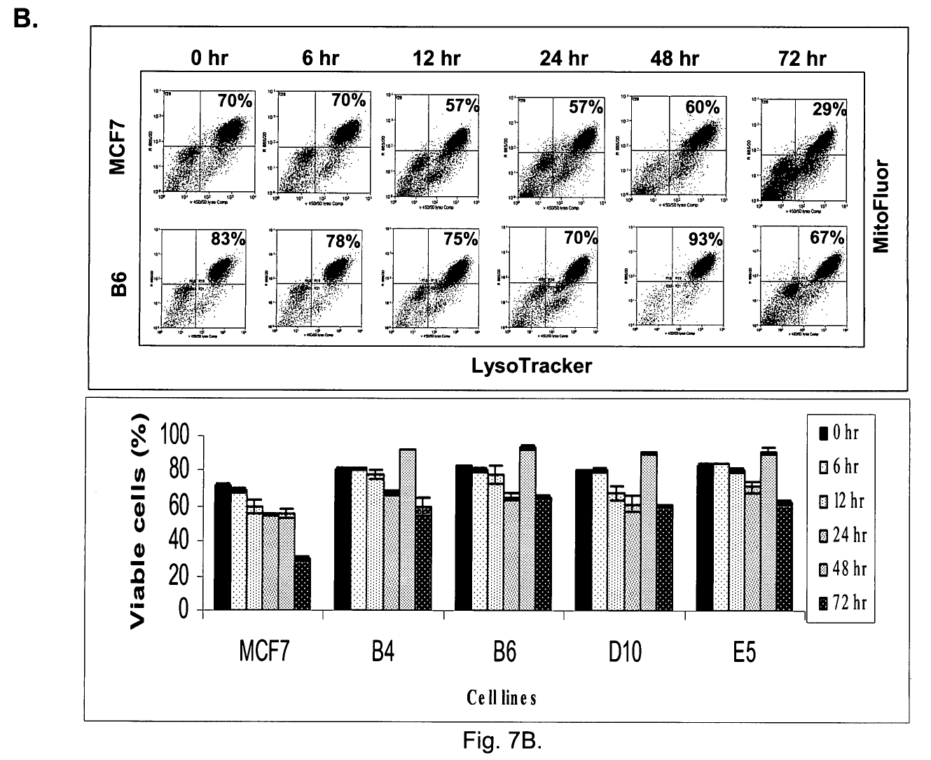

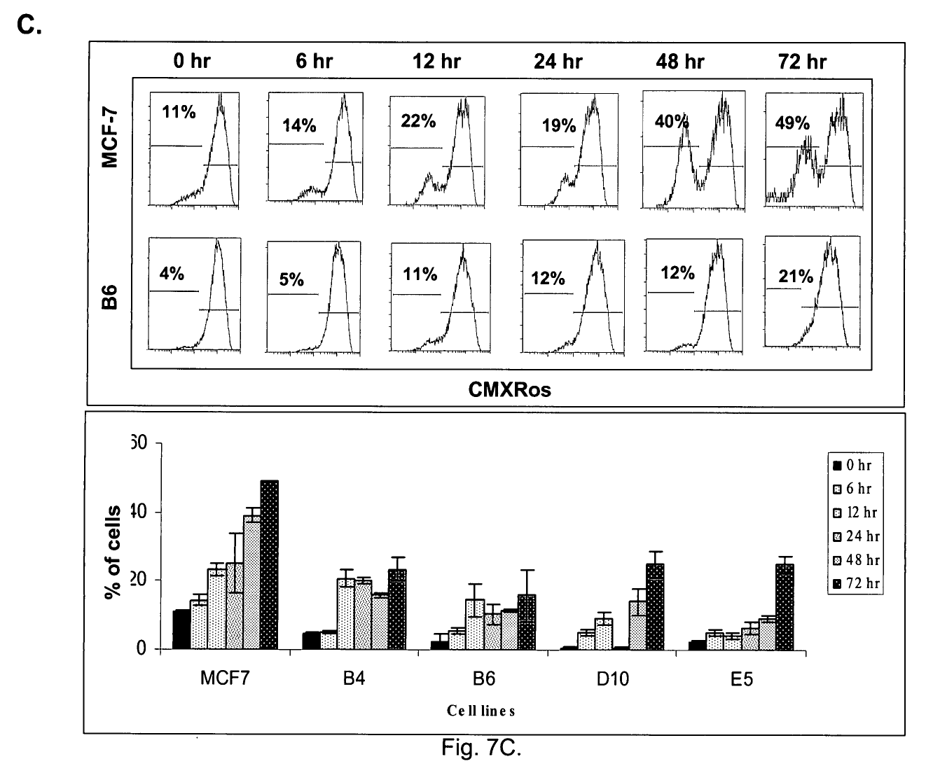

Recovery of a diverse group of cDNA inserts from a functional selection screen suggests that despite their apparent diversity their action might be concentrated within a relatively narrow functional space with the general outcome of increased survival of drug-exposed cells. A similar response to 4OHTAM was observed in all RIGs-expressing populations: reduced sensitivity to drug-induced damage (FIG. 2), a similar G1 phase block in response to drug treatment (FIG. 4), and a virtually identical functional changes (accumulation of AVO, structural and functional protection of mitochondria, and maintenance of intact plasma membrane; FIG. 7). Increased proliferation cannot explain resistance induced by RIGs (FIG. 3), although the very ability to proliferate in the presence of 4OHTAM can contribute to resistance phenotype of at least three (B4, B6, and D10) RIGs (E5 is as sensitive to proliferation block as parental cells, although this block does not result in cell death for E5-expressing cells).

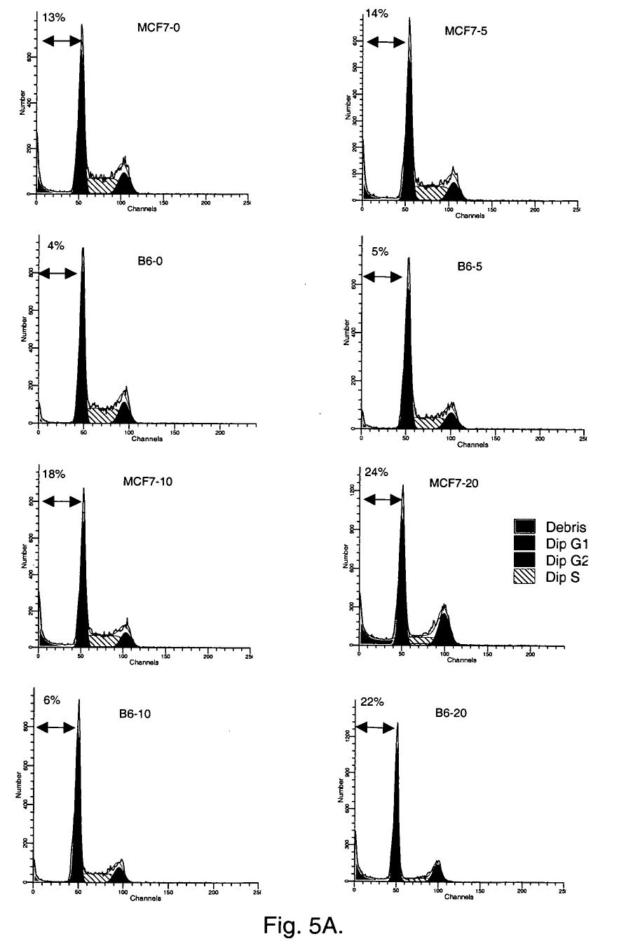

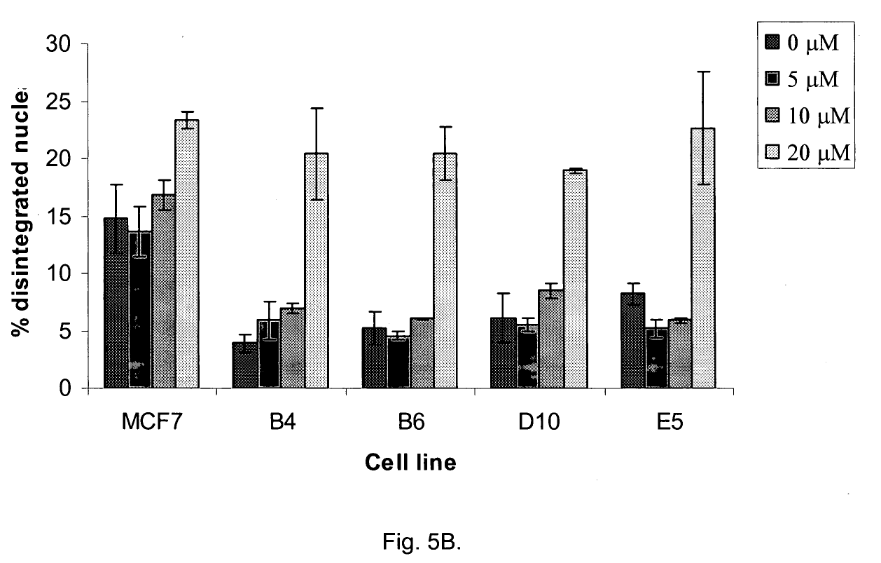

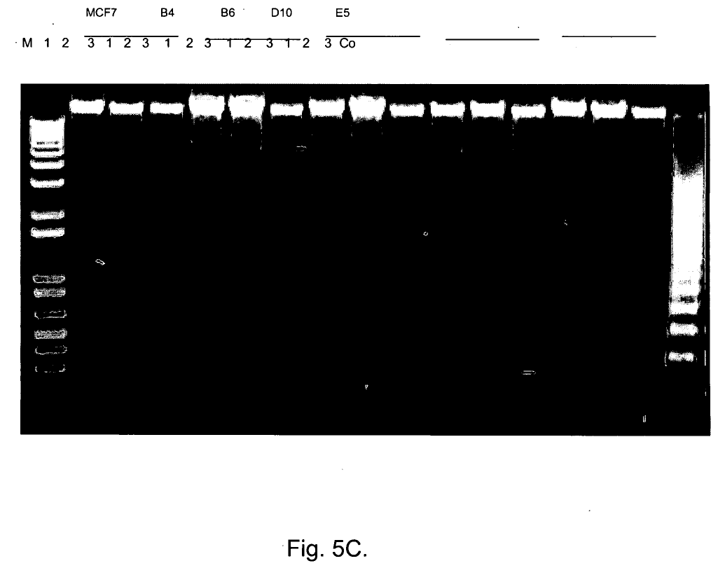

The deletion of a 47 bp fragment in caspase 3 gene (exon 3) in MCF-7 causes abnormal splicing of this pre-mRNA, which leaves out most of the exon 3, and abrogates translation of the mRNA (Janicke et al., 1998. J Biol Chem 273:9357-9360). Caspase 3 is responsible for cleavage of inhibitory DNA fragmentation factor subunit 45 (DFF-45) and release of its active counterpart DFF-40 (Inohara et al., 1999. J Biol Chem 274:270-274), it is expected that apoptosis would be completely blocked in MCF-7; however, cleavage of DFF-45 can still be detected (Janicke et al., 1998. J Biol Chem 273:15540-15545), indicating that active DFF-40 can be released, and oligonucleosomal DNA fragmentation can occur (Semenov et al., 2004. Nucleosides Nucleotides Nucleic Acids 23:831-836). In case of 4OHTAM treatment, no fragmentation or any sign of a sub-G1 peak was observed, suggesting that there was no apoptotic degradation (FIG. 5).

Type 2 physiologic cell death or autophagic cell death (APCD) is an alternative pathway of active cell death that involves encapsulation of intracellular components inside acidic vesicular organelles (AVO) and their proteolytic degradation (reviewed in (Klionsky, 2005. Autophagy. Curr Biol 15:R282-283; Edinger et al., 2004. Curr Opin Cell Biol 16:663-669; Rodriguez-Enriquez et al., 2004. Int J Biochem Cell Biol 36:2463-2472)). While excessive autodigestion is unquestionably detrimental to the cell, limited proteolysis of damaged organelles can well be a prerequisite of survival by reducing death-promoting signals (Lemasters et al., 2002. Antioxid Redox Signal 4:769-781; Edinger et al., 2003. Cancer Cell 4:422-424; Lemasters, 2005. Gastroenterology 129:351-360; Levine and Yuan, 2005. J Clin Invest 115:2679-2688). It has been shown that damaged mitochondria initiate APCD in hepatocytes (Elmore et al., 2001. Faseb J 15:2286-2287), so development of AVO and autophagic removal of such mitochondria can reduce death signaling and promote cell survival. It is contemplated that AVO is a mark of cells fighting to stay alive rather than a feature of cells destined to die.

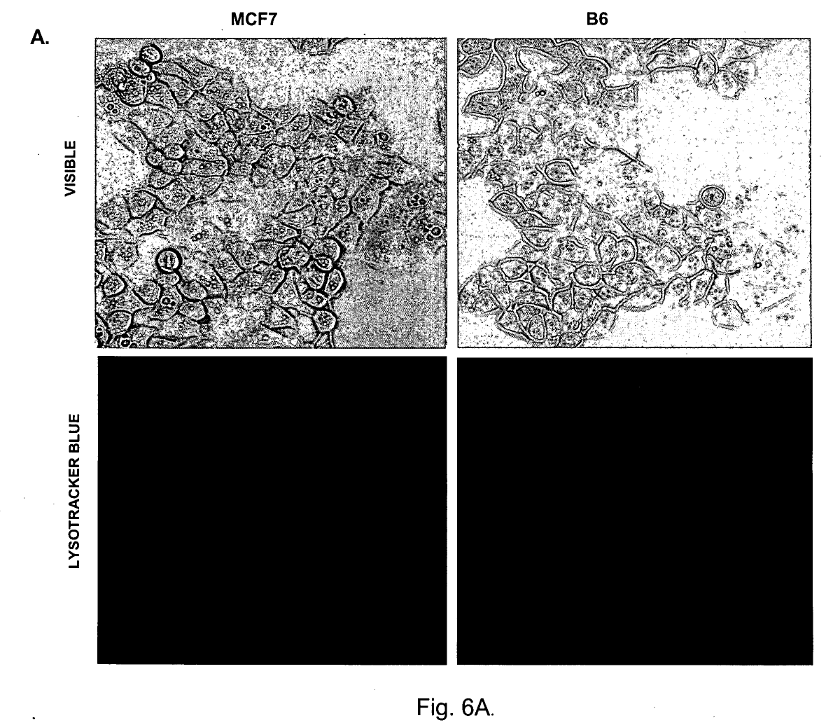

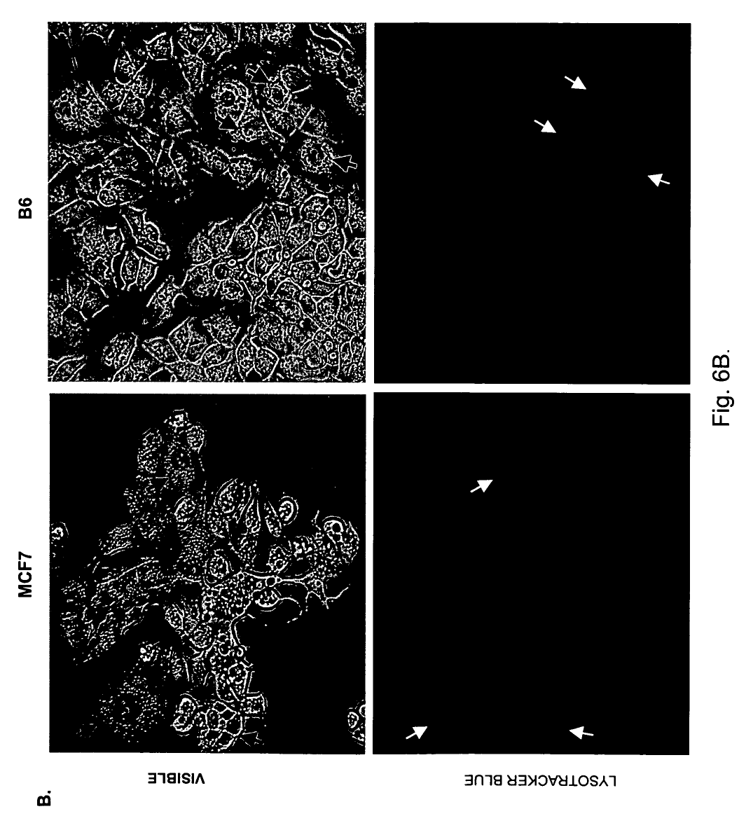

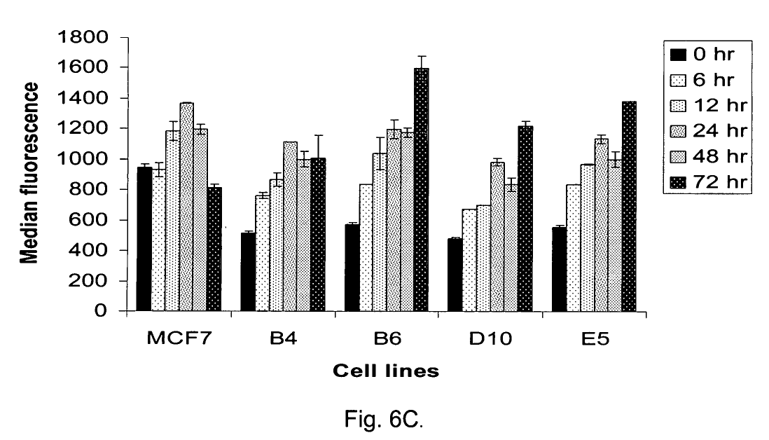

A large number of vacuoles were observed in RIGs-expressing cells treated with 4OHTAM (FIGS. 6 and 7). An understanding of the mechanism is not necessary to practice the present invention. Nonetheless, it is contemplated that a possibility of RIGs stabilizing mitochondria and thus preserving energy production in drug-treated cells can be construed from direct association of kinesin with mitochondria (Iborra et al., 2004. BMC Biol 2:9) and its role in regulation of mitochondria-dependent cell death events (Tao et al., 2005. Cancer Cell 8:49-59); from the role of MIF in inhibition of Bax and Bid cleavage, and thus in inhibition of mitochondria-dependent death pathway (Baumann et al., 2003. Faseb J 17:2221-2230); from the function of MIF as oxidoreductase and its corresponding role in reducing reactive oxygen species-induced damage (Kleemann et al., 1998. J Mol Biol 280:85-102); from hypoxia-induced inhibition of QTRT1/TGT activity (Langgut and Reisser, 1995. Nucleic Acids Res 23:2488-2491), which might cause accumulation of unmodified tRNA species in mitochondria; and from PRCP activity as a lysosomal carboxypeptidase (Odya et al., 1978. J Biol Chem 253:5927-5931), which might affect mitochondrial stability in autophagic vacuoles or even stability of vacuoles themselves.

Accordingly, in some embodiments, the present invention provides methods of regulating resistance to drug therapy by altering the expression of a resistance inducing gene including, but not limited to, macrophage migration inhibitory factor (MIF), prolylcarboxypeptidase, tRNA-guanine transglycosylase, or kinesin light chain (KNS2). In some embodiments, the present invention provides a method of monitoring resistance to chemotherapeutice treatment (e.g., Tamoxifen treatment) by measuring the levels of expression of macrophage migration inhibitory factor (MIF), prolylcarboxypeptidase, tRNA-guanine transglycosylase, or kinesin light chain (KNS2). In some embodiments, resistance is monitored by measuring the expression of two or more of these genes. In some embodiments, the present invention provides bio-markers (e.g., MIF) of pre-existing resistance to chemotherapeutic agents (e.g., tamoxifen). In some embodiments, the present invention provides biomarkers (e.g., MIF) of emerging resistance to chemotherapeutic agents (e.g., tamoxifen) in previously treated patients (e.g., in patient cells treated with tamoxifen). In some embodiments, the present invention provides methods of making cells resistant to chemotherapeutic agents by over-expressing macrophage migration inhibitory factor (MIF), prolylcarboxypeptidase, tRNA-guanine transglycosylase, or kinesin light chain (KNS2) in the cell. The present invention also provides compositions (e.g., cells over-expressing macrophage migration inhibitory factor (MIF), prolylcarboxypeptidase, tRNA-guanine transclycosylase, or kinesin light chain (KNS2)) useful for screen chemotherapeutic agents. In some embodiments, the present invention provides methods of altering expression and/or activities of the markers in vitro and/or in vivo, including, but not limited to, expression of exogenous copies of the marker (e.g., under control of an inducible promoter) or use of antibodies or siRNA molecules to inhibit marker expression or activity. Modulation of expression finds use in research, drug screening, and therapeutic applications (e.g., co-administration with known therapies).

I. Diagnostic Methods

As described above, in some embodiments, the present invention provides diagnostic methods for the detection of expression of resistance inducing genes. In some embodiments, diagnostic methods identify individuals at risk of developing resistance to chemotherapeutic drugs or that have existing resistance (e.g., so that an alternative medical route can be chosen). In other embodiments, diagnostic methods are utilized to monitor the development of drug resistance in an individual undergoing chemotherapy.

B. Detection of Markers

In some embodiments, the present invention provides methods for detection of expression of resistance inducing genes. In preferred embodiments, expression is measured directly (e.g., at the RNA or protein level). In some embodiments, expression is detected in tissue samples (e.g., biopsy tissue). In other embodiments, expression is detected in bodily fluids (e.g., including but not limited to, plasma, serum, whole blood, mucus, and urine). The present invention further provides panels and kits for the detection of markers. In preferred embodiments, the presence of a resistance inducing gene is used to provide a prognosis to a subject. The information provided is also used to direct the course of treatment. For example, if a subject is found to have a marker indicative of a resistant tumor, additional therapies (e.g., hormonal or radiation therapies) can be started at a earlier point when they are more likely to be effective (e.g., before metastasis).

The present invention is not limited to the markers described above. Any suitable marker that correlates with drug resistance may be utilized, including but not limited to, those described in the illustrative examples below. Additional markers are also contemplated to be within the scope of the present invention. For example, screening experiments using the method described in Example 1 conducted during the course of development of the present invention identified 24-dehydrocholesterol reductase (seladin) (NM_{—}014764, DHCR24); Ribosomal protein S15 (NM_{—}001018, RPS15); protective protein for beta-galactosidase (NM_{—}000308, PPGB); and Actin, gammal (NM_{—}001614, ACTG1). Any suitable method may be utilized to identify and characterize markers suitable for use in the methods of the present invention, including but not limited to, those described in illustrative Examples below.

In some embodiments, the present invention provides a panel for the analysis of a plurality of markers. The panel allows for the simultaneous analysis of multiple markers correlating with drug resistance. Depending on the subject, panels may be analyzed alone or in combination in order to provide the best possible diagnosis and prognosis. Markers for inclusion on a panel are selected by screening for their predictive value using any suitable method, including but not limited to, those described in the illustrative examples below.

1. Detection of RNA

In some preferred embodiments, detection of resistance inducing genes (e.g., including but not limited to, those disclosed herein) is detected by measuring the expression of corresponding mRNA in a tissue sample (e.g., tumor tissue). mRNA expression may be measured by any suitable method, including but not limited to, those disclosed below.

In some embodiments, RNA is detection by Northern blot analysis. Northern blot analysis involves the separation of RNA and hybridization of a complementary labeled probe.

In still further embodiments, RNA (or corresponding cDNA) is detected by hybridization to a oligonucleotide probe). A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

2. Detection of Protein

In other embodiments, gene expression of resistance inducing genes is detected by measuring the expression of the corresponding protein or polypeptide. Protein expression may be detected by any suitable method. In some embodiments, proteins are detected by immunohistochemistry. In other embodiments, proteins are detected by their binding to an antibody raised against the protein. The generation of antibodies is described below.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference.

3. Data Analysis

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of drug resistance) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

4. Kits

In yet other embodiments, the present invention provides kits for the detection and characterization of resistance inducing genes. In some embodiments, the kits contain antibodies specific for a cancer marker, in addition to detection reagents and buffers. In other embodiments, the kits contain reagents specific for the detection of mRNA or cDNA (e.g., oligonucleotide probes or primers). In preferred embodiments, the kits contain all of the components necessary to perform a detection assay, including all controls, directions for performing assays, and any necessary software for analysis and presentation of results.

II. Antibodies

The present invention provides isolated antibodies. In preferred embodiments, the present invention provides monoclonal antibodies that specifically bind to an isolated polypeptide comprised of at least five amino acid residues of the resistance inducing genes described herein. These antibodies find use in the diagnostic and therapeutic methods described herein.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a cancer marker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a cancer marker of the present invention (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

III. Drug Screening

In some embodiments, the present invention provides drug screening assays (e.g., to screen for anticancer drugs). The screening methods of the present invention utilize resistance inducing genes identified using the methods of the present invention. For example, in some embodiments, the present invention provides methods of screening for compound that alter (e.g., increase or decrease) the expression of resistance inducing genes. In some embodiments, candidate compounds are antisense or siRNA agents (e.g., oligonucleotides) directed against resistance inducing genes. In other embodiments, candidate compounds are antibodies that specifically bind to a resistance inducing gene of the present invention. In still further embodiments, candidate compounds are small molecules that alter the expression or biological activity of the resistance inducing genes.

In one screening method, candidate compounds are evaluated for their ability to alter resistance inducing gene expression by contacting a compound with a cell expressing a resistance inducing gene and then assaying for the effect of the candidate compounds on expression. In some embodiments, the effect of candidate compounds on expression of a resistance inducing gene is assayed for by detecting the level of resistance inducing gene mRNA expressed by the cell. mRNA expression can be detected by any suitable method.

In other embodiments, the effect of candidate compounds on expression of resistance inducing genes is assayed by measuring the level of polypeptide encoded by the resistance inducing genes. The level of polypeptide expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) which bind to resistance inducing genes of the present invention, have an inhibitory (or stimulatory) effect on, for example, resistance inducing gene expression or activity, or have a stimulatory or inhibitory effect on, for example, the expression or activity of a resistance inducing gene substrate. Compounds thus identified can be used to modulate the activity of target gene products either directly or indirectly in a therapeutic protocol, to elaborate the biological function of the target gene product, or to identify compounds that disrupt normal target gene interactions.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. NatI. Acad. Sci. 87:6378-6382 [1990]; Felici, J. Mol. Biol. 222:301 [1991]).

In one embodiment, an assay is a cell-based assay in which a cell that expresses a cancer marker protein or biologically active portion thereof is contacted with a test compound, and the ability of the test compound to the modulate resistance inducing gene activity is determined. Determining the ability of the test compound to modulate resistance inducing gene activity can be accomplished by monitoring, for example, changes in enzymatic activity. The cell, for example, can be of mammalian origin.

IV. Therapies

In some embodiments, the present invention provides therapies that reduce the expression or biological activity of resistance inducing genes. In some embodiments, the therapies find use in combination with existing chemotherapy regimes. In certain embodiments subjects at risk of developing drug resistance or subjects identified as a having a marker of drug resistance (e.g., identified using the diagnostic methods described herein) are treated with therapeutic agents (e.g., identified using the drug screening methods disclosed herein).

A. Antisense Therapies

In some embodiments, the present invention targets the expression of resistance inducing genes. For example, in some embodiments, the present invention employs compositions comprising oligomeric antisense compounds, particularly oligonucleotides (e.g., those identified in the drug screening methods described above), for use in modulating the function of nucleic acid molecules encoding resistance inducing genes of the present invention, ultimately modulating the amount of cancer marker expressed. This is accomplished by providing antisense compounds that specifically hybridize with one or more nucleic acids encoding resistance inducing genes of the present invention. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of cancer markers of the present invention. In the context of the present invention, “modulation” means either an increase (stimulation) or a decrease (inhibition) in the expression of a gene. For example, expression may be inhibited to potentially prevent drug resistance.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a resistance inducing gene of the present invention. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5!-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in U.S. Patent WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural intemucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their intemucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′ -5′ to 5′ -3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl intemucleoside linkages, mixed heteroatom and alkyl or cycloalkyl intemucleoside linkages, or one or more short chain heteroatomic or heterocyclic intemucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the intemucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos.: 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂—[ wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2. degree ° C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-S-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

B. RNA Interference (RNAi)

In some embodiments, RNAi is utilized to inhibit resistance inducing gene expression. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001;15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

C. Genetic Therapies

The present invention contemplates the use of any genetic manipulation for use in modulating the expression of resistance inducing genes of the present invention. Examples of genetic manipulation include, but are not limited to, gene knockout (e.g., removing the resistance inducing gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

D. Antibody Therapy

In some embodiments, the present invention provides antibodies that target resistance inducing genes. Any suitable antibody (e.g., monoclonal, polyclonal, or synthetic) may be utilized in the therapeutic methods disclosed herein. In preferred embodiments, the antibodies used for cancer therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the therapeutic antibodies comprise an antibody generated against a resistance inducing gene of the present invention, wherein the antibody is conjugated to a cytotoxic agent. In such embodiments, a tumor specific therapeutic agent is generated that does not target normal cells, thus reducing many of the detrimental side effects of traditional chemotherapy. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of endothelial cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technicium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments may include agents such as a coagulant, a cytokine, growth factor, bacterial endotoxin or the lipid A moiety of bacterial endotoxin. For example, in some embodiments, therapeutic agents will include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted tumor cells as required using known conjugation technology (See, e.g., Ghose et al., Methods Enzymol., 93:280 [1983]).

For example, in some embodiments the present invention provides immunotoxins targeting a resistance inducing genes of the present invention. Immunotoxins are conjugates of a specific targeting agent typically a tumor-directed antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In other embodiments, particularly those involving treatment of solid tumors, antibodies are designed to have a cytotoxic or otherwise anticellular effect against the tumor vasculature, by suppressing the growth or cell division of the vascular endothelial cells. This attack is intended to lead to a tumor-localized vascular collapse, depriving the tumor cells, particularly those tumor cells distal of the vasculature, of oxygen and nutrients, ultimately leading to cell death and tumor necrosis.

In preferred embodiments, antibody based therapeutics are formulated as pharmaceutical compositions as described below. In preferred embodiments, administration of an antibody composition of the present invention results in a measurable decrease in cancer (e.g., decrease or elimination of tumor).

E. Pharmaceutical Compositions

The present invention further provides pharmaceutical compositions (e.g., comprising the therapeutic or research compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more antisense compounds and (b) one or more other chemotherapeutic agents that function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other non-antisense chemotherapeutic agents are also within the scope of this invention. Two or more combined compounds may be used together or sequentially.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

Experimental

The following examples are provided in order to demonstrate and further illustrate certain preferred embodiments and aspects of the present invention and are not to be construed as limiting the scope thereof.

### EXAMPLE 1

Materials and Methods

Cells: MCF-7 (ATCC: HTB-22) cells were grown in Dulbecco's Modified Eagle's medium, 2 mM glutamine, 0.1 mM non-essential amino acids, 10 units/ml of penicillin, 10 μg/ml of streptomycin (all—Invitrogen, Calif.), supplemented with 10% fetal bovine serum (HyClone, Utah), 6 μg/ml of insulin (Sigma, Mo.), 30 μg/ml of fungin, and 10 μg/ml of plasmocin (both—InvivoGen, Calif.). 4-Hydroxytamoxifen (Sigma, Mo.) was used as 10 mM stock solution in ethanol and stored at −20° C.

cDNA expression library: VIRAPORT Fetal Human Brain full-length cDNA expression library in pFB vector was purchased from Stratagene, Calif., and amplified once on a solid support. To monitor the efficiency of retroviral infection a pFB vector with enhanced green fluorescent protein (GFP, Invitrogen, Calif.) was used.

Retroviral infection: VSVg-pseudotyped retroviral supernatant was prepared after transient transfection of 293T cells by Dr. A. Miyanohara (Program in Human Gene Therapy, UCSD, La Jolla, Calif.) using a 10:1 mixture of cDNA library- and GFP-expressing constructs. For library transduction MCF-7 cells were plated at 10⁶ per 100 mm plate 24 hr prior to infection. Polybrene (1 μg/ml final concentration) was added to viral supernatant, which was filtered through 0.45 μm filter to remove stray cells, and added to MCF-7 for 24 hr. Following infection cells were allowed to recover for 24 hr, collected and frozen in aliquots of 10⁶ cells. An aliquot was used to determine the fraction of cells that expressed GFP, and an estimate regarding library coverage was made. Reinfection experiments with individual clones were performed similarly.

Selection with 4OHTAM: cells were plated in Peel-Off tissue culture flasks (Sigma, Mo.) at 10⁶ cells per 150 cm² flask 24 hr prior to selection with 4OHTAM (20 μM final concentration); selection continued for 14 days with media replacement every two days. Surviving cells were expanded in drug-free media. The screen was performed twice with independent infections.

DNA and RNA isolation: genomic DNA was prepared using DNAeasy Tissue Kit (Qiagen, Calif.); total RNA isolation was prepared using RNAqueous-4PCR Kit (Ambion, Tex.); RT-PCR RNA samples were treated with DNase I and first DNA strand was synthesized using RETROscript kit (Ambion, Tex.). Manufacturers' protocols were followed in each case.

PCR, cloning and sequencing: Advantage-2 polymerase (Clontech, Calif.) was used for PCR (38 cycles; 94° C., 30 sec; 59° C., 20 sec; 68° C., 60 sec); vector-specific primers for insert recovery were pFB-F (CCTAGAACCTCGCTGGAAAGGACCTTACAC (SEQ ID NO:1)) and pFB-R (AGAGTCCCGCTCAGAAGAACTCGGATCG (SEQ ID NO:2)). PCR products were cloned into pGEM-T Easy vector (Promega, Wis.) and sequenced using M13 primers. The same setup was used for RT-PCR with pFB-F and gene-specific primers:

B4-R:
5′ CTGCGGCTCTTAGGCGAAGGTGGAGTTG (SEQ ID NO:3) 3′
B6-R:
5′ GGGACTTACAAATGGGCCAAAGACAC (SEQ ID NO:4) 3′
D10-R:
5′ CAATGCCAGGTCAGCCCAGTGTGATTC (SEQ ID NO:5) 3′
E5-R:
5′ AAGGTCACGCCAGCCGTGTGGTTATTAG (SEQ ID NO:6) 3′

Colony Forming Assay: five hundred cells were plated per 60 mm plate and allowed to recover overnight. The media was then replaced with 4OHTAM-containing media (7.5 μM and 10 μM); in control (untreated) plates media was replaced with drug-free media. Treatment continued for 14 days with media replacement every two days; then cells were allowed to recover for two weeks, fixed with alcohol and stained with crystal violet (2% w/v). Colonies (over 150 cells per group) were counted. Experiments were done in triplicate.

Cell staining. Propidium iodide (DNA content): cells were permeabilized with cold EtOH, incubated with propidium iodide/RNase staining buffer (BD Bioscience, Calif.) for 15 min at room temperature, and analyzed by flow cytometry.

Propidium iodide (plasma membrane integrity): one million cells were plated in 12-well culture dish, treated with 4OHTAM for specified periods of time, trypsinized, combined with floaters, resuspended in ice cold 100 μM PBS, stained with 10 μM PI/RNASE buffer (BD Bioscience, San Jose Calif.) for 15 min in the dark at room temperature, and analyzed by flow cytometry.

MitoTracker Red CMXRos (mitochondrial membrane potential) and MitoFluor 589 (mitochondrial mass detection): both dyes were obtained from Molecular Probes, Calif., and added to cells (250 nM final concentration) for 25 min at 37° C. in the CO₂ incubator. Cells were then trypsinized and analyzed by flow cytometry or by fluorescent microscope.

LysoTracker Blue DND-22 (lysosome/vacuole compartment) from Molecular Probes, Calif. was added to cells (800 nM final concentration) for 1.5 hr at 37° C. in the CO₂ incubator. Cells were then trypsinized and analyzed by flow cytometry.

Flow Cytometry was performed using a Beckman Coulter Epics XL-MCL (Beckman, Fla.) with System II v. 3.0 software and CYAN (DakoCytomation, Colo.) and Summit v. 3.3 software.

Light/Fluorescence microscopic images were acquired with Leica Microsystems DM IRB (Germany) and processed with Image PRO Plus software.

Results

1. Infection of MCF-7 Cells with cDNA Library in a Retroviral expression Vector, Selection of Resistant Clones and Identification of Integrated cDNAs in Surviving Cells.

VIRAPORT Human Fetal Brain full-length cDNA library (Stratagene) in pFB vector was chosen as the best available full-length cDNA expression library; population of mRNA in human brain is of the highest complexity (Bantle and Hahn, 1976. Cell 8:139-150) representing the majority of expressed genes (Takahashi, 1992. Prog Neurobiol 38:523-569). The library was amplified on solid support, and plasmid DNA was isolated using standard column technique (Qiagen). pFB vector does not contain a marker, so a pFB-EGFP construct was created, and VSVg-pseudotyped supernatant was produced using a 10:1 mixture of library-containing plasmid and pFB-EGFP. Test infections of MCF-7 indicated that up to 20% (12-20% for different batches of supernatant) of cells expressed EGFP after a single infection; to calculate the number of cells required for selection we assumed a 50-75% infection rate with library constructs. The library contained 2×10⁶ primary clones (Stratagene); assuming no losses during amplification and production of supernatant, we infected 8×10⁶ MCF-7 cells to achieve at least two-fold library coverage at 50% infection rate. No noticeable cell death was observed after the infection; for selection cells were plated using Peel-Off tissue culture flasks at 10⁶ cells per 150 cm² flask. Selection with 4OHTAM, and recovery and expansion of surviving clones were done as described in Materials and Methods. Screening was repeated twice using three independent batches of viral supernatant for each screen.

Surviving clones (19 from the first screen, and 25—from the second) were individually expanded; their genomic DNA was isolated, and used for PCR with vector-specific primers (FIG. 1A). PCR results indicate that in many cases selected clones contain at least two different proviruses including EGFP-containing marker (FIG. 1B). Several inserts were isolated, and four of them were chosen for further investigation (Table 1): clone B4 (macrophage migration inhibitory factor, MIF), clone B6 (prolylcarboxypeptidase, PRCP), clone D10 (tRNA-guanine transglycosylase, QTRT1/TGT) and clone E5 (kinesin light chain, KLC1G/KSN2). Complete open reading frames (ORFs) were present in clones B4, B6, and D10, while E5 contained a 5′ truncation (FIG. 1C).

2. Re-Introduction of Selected Genes Induces Resistance to 4OHTAM into Naïve MCF7 Cells.

To confirm that resistance to 4OHTAM is caused by overexpression of B4, B6, D10 and E5 as opposed to drug-induced genomic alterations (a mutation or a change in expression of an endogenous gene), selected cDNAs were cloned into the original pFB vector and transduced into naïve MCF-7 cells with individual constructs and tested for resistance to 4OHTAM. Each population now contained only one type of selected cDNA (FIG. 2A; note that genomic DNA from an infected population rather than DNA from a single-cell clone was used for PCR in this case). Infection rate again was determined by adding pFB-EGFP to the corresponding plasmid (1:10 ratio; note the presence of EGFP-specific band in FIG. 2A) and by assessing the percentage of EGFP-expressing cells in each infected population. Expression of delivered cDNAs was confirmed in RT-PCR experiments (FIG. 2A.2) using a combination of one vector-specific and one gene-specific primer (see Materials and Methods). Both incorporation and expression of B4 insert was significantly weaker than that of other inserts.

Resistance was determined by colony-forming assay as described in Materials and Methods: plates were stained, and colonies were counted (FIG. 2B); results of the experiment were plotted (FIG. 2C). The most pronounced difference between control (EGFP-only) and cDNA-expressing cells was seen with 7.5 μM 4OHTAM (FIG. 2C) when cDNA-expressing cells were five-six times more resistant to the drug. In heterogeneous populations the level of resistance is lower than in single-cell clones (presence of cells without inserts, different levels of expression, etc), so resistance induced by selected cDNAs in individual cells can be much higher. As selected cDNAs induced resistance to 4OHTAM, they were considered to be resistance-inducing genes (RIGs).

3. Changes of Growth Characteristics and Increased Viability of Cells in RIGs-Expressing Populations.

To gain a better understanding of changes induced by the RIGs, cell growth of RIGs-expressing populations was evaluated with and without 4OHTAM (FIG. 3). In drug-free media the proliferation rate of the B4 RIG-expressing cells was at least equal to parental MCF-7 cells or GFP-expressing control, while for B6, D10 and E5 the proliferation rate was higher (FIG. 3A). Growth-inhibiting concentration of 4OHTAM reduced growth of parental, control and the RIGs-expressing cells to a similar degree (FIG. 3B) suggesting that the estrogen receptor pathway was functional in resistant cells; further increase of the drug concentration did not block growth of cells expressing RIGs B4, B6, and D10, while growth of cells with RIG E5 was essentially stopped (FIG. 3C). Continued incubation in drug-free media resulted in massive cell death for control and parental cells, while RIG E5-expressing cells recovered and continued growth. It is contemplated that cessation of growth for parental and control cells reflects terminal damage and initial stages of cellular demise, whereas for RIG E5-expressing cells cessation of growth is a protective response to drug exposure.

No gross differences were apparent in the distribution of cells in the cell cycle when tested in drug-free media (FIG. 4A). Drug treatment, however, caused a noticeable increase in the GI content with a concurrent decrease in the S content for all RIGs-expressing cells as early as 12 hr after beginning of drug treatment compared with parental MCF-7 (FIG. 4B), suggesting that RIGs expression triggered and maintained a stronger G1 delay in response to drug; such a delay is consistent with cytostatic effect of antiestrogens (Taylor et al., 1983. Cancer Res 43:4007-4010; Reddel et al., 1985. Cancer Res 45:1525-1531) and confirms functional activity of estrogen receptor in RIGs-expressing cells. All four RIGs act in a similar way. Expression of RIGs reduced accumulation of cellular debris with DNA content below G1 (FIGS. 4A and 4B, bottom panel), suggesting that RIGs improve cell viability both in the presence and in the absence of the drug. The relative amount of MCF-7 debris decreases with accumulation of cells in G1 and decline of cells in S phase, which may reflect increased stability of parental cells in G1 compared to S phase. Similar changes are much less pronounced for the RIGs-expressing cells, suggesting that their stability is sufficiently high, and G1 delay does not improve it further.

The presence of cell debris with DNA content below G1 suggested that cell death induced by 4OHTAM could proceed by apoptosis. No evidence was found of a characteristic sub-G1 peak associated with apoptosis in FACS profiles (FIG. 5A) even when very high concentrations of 4OHTAM (20 μM) caused destruction not only of parental but of RIGs-containing cells as well (FIGS. 5A and 5B). Similarly no characteristic DNA fragmentation pattern (DNA ladder) was detected when genomic DNA was analyzed (FIG. 5C); it was concluded that apoptosis was not the mechanism of cell death for either parental cells or the RIGs-expressing derivatives.

Quantitation of cell debris shows that protection afforded by the RIGs is not effective when high levels of 4OHTAM (20 μM) are used (FIGS. 5A and 5B).

4.What Type of Cell Death is Influenced by RIGs

Apoptosis was initially considered as the mechanism of cell death induced by 4OHTAM, but the data (FIG. 5) suggested that another type of cell demise was most probably involved. Careful examination of cell death induced in MCF-7 by TAM in the presence of estrogen led Bursch et al to conclude that autophagic cell death (APCD) was involved (Bursch et al., 1996. Carcinogenesis 17:1595-1607), so several key elements of APCD development in MCF-7 cells were tested in the RIGs-expressing populations.

Formation of acidic autophagic vacuoles (also called acidic vesicular organelles, AVO) is one of the morphological features of the APCD (Bursch et al., 2000. Ann N Y Acad Sci 926:1-12; Scarlatti et al., 2004 J Biol Chem 279:18384-18391), and such vacuoles stainable with acidic dye LysoTracker Blue DND-22 appeared in MCF-7 cells treated with 4OHTAM (FIGS. 6A and B). Morphologically similar vacuoles were visible in all RIGs-expressing 4OHTAM-treated cells as well (images shown for B6; FIG. 6B). When cells were stained with LysoTracker Blue DND-22 and median fluorescence determined by FACS was plotted (FIG. 6C), all RIGs-containing cells accumulated much less dye than parental cells. Upon drug exposure an increase in fluorescence of all RIGs-expressing cells was observed as early as 6 hr after drug exposure, while in MCF7 cell a similar increase was delayed to 12 hr. Fluorescence reached comparable level in all cells after 24 hr exposure to 4OHTAM, and then declined in MCF-7 cells, while in RIGs-expressing cells it remained high. Expression of the RIGs does not prevent formation of AVO stainable by LysoTracker Blue DND-22, and suggests that AVO per se do not define APCD, which can be blocked downstream of vacuole formation.

To explore this effect further different fluorescent dyes were used to study morphological changes and alterations in mitochondrial function in cells treated with 4OHTAM: LysoTracker Blue DND-22—to follow appearance and development of AVO; propidium iodide (PI)—to evaluate changes in permeability of the plasma membrane; MitoFluor 589—to access alterations in mitochondrial mass; and MitoTracker Red CMXRos—to measure mitochondrial activity. All RIGs-expressing cells produced very similar profiles (representative data is shown for B6; quantitation of FACS data for all RIGs; FIG. 7), suggesting that all RIGs interfered with the same set of cellular processes.

Double staining with LysoTracker and PI was done to evaluate cells where increase in AVO correlated with increased permeability of the plasma membrane (FIG. 7A, top panel). Low PI stained cells fall into two groups, with high (lower right quadrant) and low LysoTracker staining (lower left quadrant). According to side scatter plot the lower left quadrant contains cell debris that cannot be stained with PI, while the lower right quadrant contains intact cells, so to compare potentially live cells (structurally intact and PI-impermeable) cell fraction in each lower right quadrant was plotted (FIG. 7A, lower panel): all RIGs-expressing populations contained a high fraction of live cells according to this criteria.

High LysoTracker staining does not necessarily presage cell demise: while cells with high LysoTracker staining gradually diminish in the population of MCF-7 cells, which is consistent with their eventual death, for B6 RIG-expressing cells that are resistant to the treatment (FIG. 2B), the major part of the population still stains efficiently with LysoTracker, with vast majority of cells highly positive for this stain (FIG. 7A). While a positive correlation between AVO and cell death (increase in AVO acting to promote death) can be hypothesized for MCF-7, B6 RIG-expressing cells suggest that a negative correlation might also be possible (increase in AVO acting to prevent death).

Double staining with MitoFluor 589 and LysoTracker Blue DND-22 was done in an attempt to determine a positive or negative correlation. Results of the experiment indicate that cells staining highly for mitochondria are also high in AVO (FIG. 7B, top pane, upper right quadrant), while cells with reduced mitochondrial content (dying cells) stain lower for both mitochondria and AVO (FIG. 7B, top panel, lower left quadrant), suggesting that cells with high level of AVO are more resistant.

The fate of mitochondria was also examined in experiments assessing mitochondrial activity (FIG. 7C). Dramatic increase in parental cell disintegration after 72 hr of drug treatment (FIG. 7A) correlates well with decline in cells with normal mitochondrial mass (FIG. 7B) and with accumulation of inactive mitochondria (FIG. 7C, upper panel, CMXRos). In MCF-7 a subpopulation of inactive mitochondria appears after 48 hr of drug treatment (FIG. 7C, CMXRos panel), while their physical disruption is largely delayed till 72 hr (FIG. 7B) suggesting that functional inactivation paves the way to structural demise. The intracellular content of this organelle on a per cell basis remains fairly constant with only insignificant fluctuations (FIG. 7B), indicating a tight control of the number of mitochondria per cell. 1S A population of B6-containing cells treated with 4OHTAM for 72 hr contains approximately 30% of cells with reduced mitochondrial content (67% have normal mitochondrial content), which is close to the distribution of parental MCF-7 cells treated for 48 hr (FIG. 7B). At the same two timepoints mitochondrial activity distributions are vastly different: a separate low-activity peak for MCF-7 and a barely noticeable asymmetry (“shoulder”) for B6-cells (FIG. 7C). The present invention is not limited to a particular mechanism. Indeed, an understanding of the mechanism is not necessary to practice the present invention. Nonetheless, it is contemplated that a possible explanation might involve a narrow range of mitochondrial activity adjustment in MCF-7, so that even a partial elimination of mitochondria leads to noticeably lower level of oxidative phosphorylation, while in B6 a wider range of activity adjustment is possible, so reduced number of mitochondria per cell does not curtail energy production.

One result of the characterization of RIGs is the similarity of their effects on cell growth (FIG. 3) cell cycle distribution (FIG. 4), and stabilization of parental cells in the presence of the drug (FIGS. 5, 6, 7). It is contemplated that these effects can be explained by modification of the same cell death pathway.

TABLE 1
cDNA inserts recovered from MCF-7 clones after selection with 4OHTAM.

| Clone | Accession | | | Cellular process and function |
|---|---|---|---|---|
| ID | number | Symbol | Description | (NCBI) |
| B4 | NM_002415 | MIF | Macrophage | Cell proliferation, cell surface |
| | | | migration | receptor linked signal transduction, |
| | | | inhibitory | inflammatory response, negative |
| | | | factor | regulation of apoptosis, |
| | | | | prostaglandin biosynthesis, |
| | | | | regulation of macrophage activity, |
| | | | | localized to extracellular region |
| B6 | NM_005040 | PRCP | Prolylcarboxy- | Lysosomal Pro-X carboxypeptidase |
| | | | peptidase | activity, serine-type peptidase |
| | | | (angiotensinase | activity, a prekallikrein activator, |
| | | | C) | localized to lysosome. |
| D10 | AK055216 | QTRT1/ | tRNA- guanine | Queuosine biosynthesis, tRNA |
| | | TGT | transglyco- | processing, queuine tRNA- |
| | | | sylase fetal | ribosyltransferase activity, localized |
| | | | brain sequence | to ribosome |
| ES | BK001170 | KLC1G/ | Kinesin light | Microtubule motor activity, kinesin |
| | | KSN2 | chain | complex |

We claim:

1. A method of detecting efficacy of chemotherapeutic agents, said method comprising detecting the expression or activity of a marker selected from the group consisting of macrophage migration inhibitory factor (MIF), prolylcarboxypeptidase, tRNA-guanine transglycosylase, and kinesin light chain (KNS2).

2. The method of claim 1, wherein said chemotherapeutic agent is tamoxifen.

3. The method of claim 1, wherein said chemotherapeutic agent is 4-hydroxytamoxifen.

4. A method of monitoring chemotherapeutic treatment, said method comprising measuring the expression of a resistance inducing gene selected from the group consisting of macrophage migration inhibitory factor (MIF), prolylcarboxypeptidase, tRNA-guanine transglycosylase, and kinesin light chain (KNS2) in a sample obtained from a subject undergoing chemotherapy.

5. The method of claim 4, wherein said measuring the expression of a resistance inducing gene comprising exposing said sample to a nucleic acid complementary to said resistance inducing gene.

6. The method of claim 4, wherein said measuring the expression of a resistance inducing gene comprising exposing said sample to a antibody that specifically binds to a polypeptide encoded by said resistance inducing gene.

7. The method of claim 4, wherein said chemotherapeutic treatment is selected from the group consisting of tamoxifen and 4-hydroxytamoxifen.

8. A method of screening compounds, comprising:
a) providing a cell expressing a a resistance inducing gene selected from the group consisting of macrophage migration inhibitory factor (MIF), prolylcarboxypeptidase, tRNA-guanine transglycosylase, and kinesin light chain (KNS2); and
b) exposing said cell to a test compound.

9. The method of claim 8, further comprising the step of measuring the effect of said test compound on the level of expression of said resistance inducing gene.

10. The method of claim 8, wherein said test compound is selected from the group consisting of an antisense nucleic acid complementary to said resistance inducing gene, an siRNA complementary to said resistance inducing gene, an antibody that specifically hybridizes to a polypeptide encoded by said resistance inducing gene, and a small molecule therapeutic.

11. The method of claim 8, wherein said cell is in vitro.

12. The method of claim 8, wherein said cell in in vivo.

13. The method of claim 12, wherein said cell is in a non-human mammal.


SEQUENCE LISTING (12 sequences)

SEQ ID NO: 1 - DNA, 30 bp, Artificial Sequence
  Synthetic
cctagaacctcgctggaaaggaccttacac

SEQ ID NO: 2 - DNA, 28 bp, Artificial Sequence
  Synthetic
agagtcccgctcagaagaactcggatcg

SEQ ID NO: 3 - DNA, 28 bp, Artificial Sequence
  Synthetic
ctgcggctcttaggcgaaggtggagttg

SEQ ID NO: 4 - DNA, 26 bp, Artificial Sequence
  Synthetic
gggacttacaaatgggccaaagacac

SEQ ID NO: 5 - DNA, 27 bp, Artificial Sequence
  Synthetic
caatgccaggtcagcccagtgtgattc

SEQ ID NO: 6 - DNA, 28 bp, Artificial Sequence
  Synthetic
aaggtcacgccagccgtgtggttattag

SEQ ID NO: 7 - DNA, 49 bp, Homo sapiens
gcctctgcgcgggtctcctggtccttctgccatcatgccgatgttcatc

SEQ ID NO: 8 - DNA, 44 bp, Homo sapiens
cacccgcactgcagtctccagcctgagccatgggccgccgagcc

SEQ ID NO: 9 - DNA, 24 bp, Homo sapiens
gttcttcaacaccagacttcagat

SEQ ID NO: 10 - DNA, 21 bp, Homo sapiens
ttgggtatgttgtggataggg

SEQ ID NO: 11 - DNA, 12 bp, Homo sapiens
atgtccacaatg

SEQ ID NO: 12 - DNA, 18 bp, Homo sapiens
gaggacaaagacactgat